(19) United States
(12) Patent Application Publication
(10) Pub. No.: US 2023/0330089 A1
(43) Pub. Date: Oct. 19, 2023

(54) COVID PREVENTION AND TREATMENT USING AN INTEGRIN INHIBITOR

(71) Applicants: BROWN UNIVERSITY, Providence, RI (US); Rhode Island Hospital (Lifespan), Providence, RI (US); Universidad Catolica del Maule, Talca (CL)

(72) Inventors: Wafik S. El-Deiry, Barrington, RI (US); Kelsey Huntington, Providence, RI (US); Lindsey Carlsen, Providence, RI (US); Olin Liang, Providence, RI (US); Matthias Piesche, Maule (CL)

(21) Appl. No.: 18/134,636

(22) Filed: Apr. 14, 2023

Related U.S. Application Data
(60) Provisional application No. 63/331,331, filed on Apr. 15, 2022.

(51) Int. Cl. A61K 31/506; A61P 31/14

(52) U.S. Cl. CPC A61K 31/506; A61P 31/14

(57) ABSTRACT

A method of treating or preventing COVID-19 in a subject in need thereof is described. The method includes administering to the subject a therapeutically effective amount of an integrin inhibitor.

### CROSS-REFERENCE TO RELATED APPLICATIONS

This application claims priority to U.S. Provisional Application Ser. No. 63/331,331, filed on Apr. 15, 2022, which is hereby incorporated by reference in its entirety.

### STATEMENT REGARDING FEDERALLY SPONSORED RESEARCH

This invention was made with government support under Grant Numbers P20GM119943 and 5P30GM122732-05, awarded by the National Institutes of Health. The Government has certain rights in this invention.

### BACKGROUND

Severe acute respiratory syndrome coronavirus 2 (SARS-CoV-2), the virus responsible for COVID-19, enters cells via interaction of its spike protein with angiotensin-converting enzyme 2 (ACE2), and possibly other receptors such as CD147/26, on human cells. Kuhn et al., Cell Mol Life Sci., 61(21):2738-43 (2004). The receptor-binding domain of spike contains upstream from the ACE2 binding site a novel RGD (Arg-Gly-Asp) motif that is absent in SARS-1. The RGD motif was originally identified within several extracellular matrix proteins as the minimal peptide sequence required for cell attachment via integrins, which make transmembrane connections to the cytoskeleton and activate many intracellular signaling pathways. Integrins are also commonly used as receptors by many human viruses. Hussein et al., Arch Virol., 160(11):2669-81 (2015). The conservation of the motif and its localization in the receptor-binding region of the SARS-CoV-2 spike protein suggests that integrins may serve as alternative receptors for this virus. Indeed, recent evidence suggests that this motif allows SARS-CoV-2 binding to integrins on human cells, facilitating viral infection (Simons et al., Sci Rep. 2021; 11:20398; FIG. 1), which may contribute to the higher transmission efficiency compared to SARS-1. There are eight known RGD-binding integrins with potential to impact on the pathogenesis of SARS-CoV-2: αvβ1, αvβ3, αvβ5, αvβ6, αvβ8, α5β1, α8β1, and αIIbβ3. A recent study has shown that blocking integrin αVβ3 prevents SARS-CoV-2 from binding to the vascular endothelium, potentially inhibiting virus-induced loss of endothelial barrier integrity and spread of SARS-CoV-2 to other organs. Nader et al., PLoS One, 16(6):e0253347 (2021).

Although several highly effective vaccines have now been developed against coronavirus disease 2019 (COVID-19), its threat to public health persists due to the presence of breakthrough cases, the current improbability of achieving herd immunity, reluctance to vaccinate among significant segments of the population, less available vaccines in much of the developing world, and the emergence of highly transmissible and immune evasive Delta and Omicron variants. Therefore, novel treatment strategies are urgently required to prevent severe disease, hospitalization and death, especially in vulnerable populations such as the elderly and immunocompromised, as well as those with pre-existing conditions including patients with cancer, or who cannot get vaccinated.

### SUMMARY OF THE INVENTION

The inventors used the pan-integrin inhibitor GLPG-0187 to demonstrate blockade of SARS-CoV-2 pseudovirus infection of target cells. Pseudotyped viruses that lack certain gene sequences of the virulent virus are safer and can be investigated in biosafety level 2 laboratories, providing a useful virological tool for the study of SARS-CoV-2. Chen M., and Zhang, Xian-En, Int J Biol Sci., 17(6): 1574-1580 (2021). Omicron pseudovirus infected normal human small airway epithelial (HSAE) cells significantly less than D614G or Delta variant pseudovirus, and GLPG-0187 effectively blocked SARS-CoV-2 pseudovirus infection in a dose-dependent manner across multiple viral variants. GLPG-0187 inhibited Omicron and Delta pseudovirus infection of HSAE cells more significantly than other variants. Pre-treatment of HSAE cells with MEK inhibitor (MEKi) VS-6766 enhanced inhibition of pseudovirus infection by GLPG-0187. Because integrins activate TGF-β signaling, we compared plasma levels of active and total TGF-β in COVID-19+ patients. Plasma TGF-β1 levels correlated with age, race, and number of medications upon presentation with COVID-19, but not with sex. Total plasma TGF-β1 levels correlated with activated TGF-β1 levels. In our preclinical studies, Omicron infects lower airway lung cells less efficiently than other COVID-19 variants. Moreover, inhibition of integrin signaling prevents SARS-CoV-2 Delta and Omicron pseudovirus infectivity, and may mitigate COVID-19 severity through decreased TGF-β1 activation.

### BRIEF DESCRIPTION OF THE FIGURES

The present invention may be more readily understood by reference to the following figures, wherein:

FIG. 1 provides a schematic representation showing Integrins mediate SARS-CoV-2 infection and activation of TGF-β in human cells, facilitating viral pathogenesis. In addition to an ACE2-binding site, SARS-CoV-2 spike protein contains an RGD motif that binds RGD integrins, allowing viral infection (left). RDG-binding integrins are a major regulator of activation of latent TGF-0, which can be blocked via integrin inhibition (right). Active TGF-β may lead to more severe COVID-19 pathogenesis. GLPG-0187 is a pan-integrin inhibitor of the integrins (shown in gray text) and has the potential to mitigate TGF-β mediated disease severity.

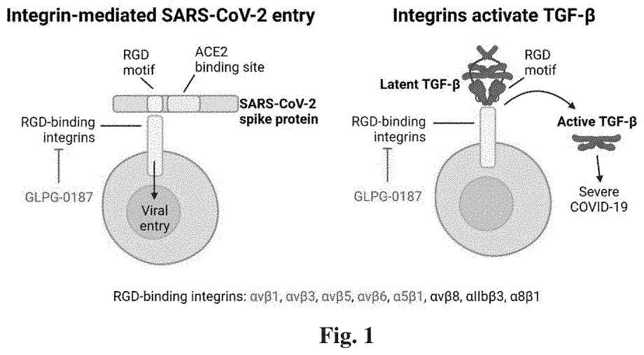

FIGS. 2A-2E provide graphs showing GLPG-0187 inhibits infection of SARS-CoV-2 pseudovirus variants D614, D614G, N501Y, E484K, N+E, NEK, R685A, Beta, Delta, and Omicron in small airway epithelial cells. (A) Treatment with 20 nM, 100 nM, 200 nM, or 1 μM GLPG-0187 for 2 hours inhibits infection by the D614G pseudovirus variant (24 hour infection time) in small airway epithelial cells compared to the VsVg positive control in a dose-dependent manner (B) Treatment with 1 μM GLPG-0187 for 3 hours inhibits infection by the D614, D614G, N501Y, E484K, N+E, NEK, R685A pseudovirus variants (20 hour infection time). (C) Treatment with 1 μM or 2 μM GLPG-0187 for 2 hours inhibits infection by the D614G, Beta, and Delta pseudovirus variants (20 hour infection time). (D) Differential rates of infectivity across D614G, Delta, and Omicron variants observed after cells were spin-infected with the same amount of pseudovirus particles (1.0×10⁶ transduction units (TU) per 1×10⁵ cells/well). (E) Treatment with 1 μM GLPG-0187 for 2 hours inhibits infection by the Omicron pseudovirus variant (26 hour infection time).

FIG. 3 provides graphs showing the MEK inhibitor VS-6766 enhances the inhibition of SARS-CoV-2 pseudovirus infection by integrin inhibitor GLPG-0187 in small airway epithelial cells. Treatment with 5 μM VS-6766 for 24 hours or with 1 μM GLPG-0187 for 3 hours inhibits infection by the D614G pseudovirus variant (20 hour infection time) in small airway epithelial cells compared to the VsVg positive control. Combination treatment involved 24 hour pre-treatment with VS-6766 followed by an additional 3 hours of treatment with GLPG-0187.

FIG. 4A-4G provide graphs showing plasma TGF-β1 levels correlate with age, race, and number of mediations administered upon presentation with COVID to the ED, but not with sex. Total TGF-β1 levels were detected in activated plasma samples. TGF-β1 plasma concentration correlation with (A) age, (B) race, (C) number of mediations administered upon presentation with COVID-19 to the emergency department (ED), (D) number of symptoms reported upon presentation to the ED, (E) sex, or (G) COVID-19 severity score. (G) COVID-19 severity score (CSS) legend. Sample values are reported in pg/mL (n=81 samples).

FIGS. 5A-5D provide graphs showing Active plasma TGF-β1 levels correlate with total TGF-β1 levels. Active TGF-β1 levels were detected in non-activated plasma samples. TGF-β1 plasma concentration correlation with (A) age, (B) race, (C) sex, or (D) COVID-19 severity score (CSS). Sample values are reported in pg/mL (n=81 samples).

### DETAILED DESCRIPTION OF THE INVENTION

The present invention provides a method of treating or preventing COVID-19 in a subject in need thereof. The method includes administering to the subject a therapeutically effective amount of an integrin inhibitor, such as GLPG-0187.

Unless otherwise defined, all technical and scientific terms used herein have the same meaning as commonly understood by one of ordinary skill in the art to which these exemplary embodiments belong. The terminology used in the description herein is for describing particular exemplary embodiments only and is not intended to be limiting of the exemplary embodiments. As used in the specification and the appended claims, the singular forms “a,” “an,” and “the” are intended to include the plural forms as well, unless the context clearly indicates otherwise. All publications, patent applications, patents, and other references mentioned herein are incorporated by reference in their entirety.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

A “subject,” as used herein, can be any animal, and may also be referred to as the patient. Preferably the subject is a mammal, such as a research animal (e.g., a monkey, rabbit, mouse or rat) or a domesticated farm animal (e.g., cow, goat, horse, pig) or pet (e.g., dog, cat). In some embodiments, the subject is a human.

“Treat”, “treating”, and “treatment”, etc., as used herein, refer to any action providing a benefit to a subject at risk for or afflicted with a condition or disease such as COVID-19, including improvement in the condition through lessening or suppression of at least one symptom, delay in progression of the disease, prevention or delay in the onset of the disease, etc.

“Preventing,” as used herein, refers to any action that decreases the risk that a subject will develop an infection by SARS-CoV-2, or that will decrease the risk of symptoms of COVID-19 should an infection nonetheless occur. Preventing infection can be done in subjects who have an increased risk of developing an infection. Subjects can have an increased risk of developing an infection as a result of, for example, being immunosuppressed, elderly, or having recently been exposed to other individuals who are infected.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses.

All scientific and technical terms used in the present application have meanings commonly used in the art unless otherwise specified. The definitions provided herein are to facilitate understanding of certain terms used frequently herein and are not meant to limit the scope of the present application.

### Treating or Preventing COVID-19

In one aspect, the present invention provides a method of treating or preventing COVID-19 in a subject in need thereof, comprising administering to the subject a therapeutically effective amount of the integrin inhibitor.

COVID-19 (Coronavirus Disease of 2019), the disease caused by severe acute respiratory syndrome coronavirus 2 (SARS-CoV2), can affect the upper respiratory tract (sinuses, nose, and throat) and the lower respiratory tract (windpipe and lungs). The lungs are the organs most affected by COVID-19 because the virus accesses host cells via the enzyme angiotensin-converting enzyme 2 (ACE2), which is most abundant in type II alveolar cells of the lungs. The virus uses a special surface glycoprotein called a “spike” (peplomer) to connect to ACE2 and enter the host cell, although additional cell surface receptors for COVID-19 entry into cells are also present. SARS-CoV-2 is a positive-sense single-stranded RNA virus. A number of variants of SARS-CoV-2 are known, including alpha, beta, gamma, delta, and omicron. In some embodiments, the COVID-19 is caused by Omicron or Delta SARS-CoV-2 infection.

The inventors have shown that SARS-CoV-2 pathogenesis can lead to a variety of changes in cytokine and chemokine profiles in patent samples, and that these changes are associated with disease severity. For example, the inventors have shown that COVID-19 disease severity is associated with macrophage activation syndrome. In addition, the chronic immune response observed with SARS-CoV-2 pathogenesis is believed to be mediated by transforming growth factor beta 1 (e.g., TGF-β1). Because integrins can activate TGF-β, in some embodiments, the subject has increased TGF-β1 levels.

Because it has been previously shown that the chronic immune response observed with SARS-CoV-2 is mediated by TGF-β1, the inventors sought to compare the levels of TGF-β1 in plasma samples from COVID (+) patients upon admission to the emergency department. They chose to focus on TGF-β1, as opposed to TGF-02 and 3, since it had been previously shown that SARS-CoV-2 infection increased TGF-β1 expression in human epithelial cells and was a driver of lung fibrosis. The inventors analyzed the levels of total TGF-β1 in COVID (+) plasma samples and found a significant correlation between TGF-β1 concentration (pg/mL) and age. They also found significant variations in TGF-β1 concentrations depending on the patient's self-reported race or ethnicity, with notably higher levels of the growth factor in White and Hispanic or Latino populations, and notably lower levels in Black and Asian or Pacific Islander populations. Patients were grouped by the number of medications they received upon disease presentation to the emergency department. Medications reported included ibuprofen, acetaminophen, bronchodilators (e.g., Albuterol), steroids (e.g., Prednisone), azithromycin, hydroxychloroquine, antibiotic, or other. A statistically significant decreased plasma TGF-β1 concentrations was noted in patients that received 2-4 medications in the Emergency Department (ED), as compared to patients who received 0-1. Next, patients were grouped by number of symptoms self-reported upon admission to the ED and noted a positive trend between TGF-β1 levels and the number of symptoms, although not significant. When comparing TGF-β1 levels between male and female, the inventors did not note a significant difference.

The inventors compared TGF-β1 levels in patients based on our COVID-19 Severity Score (CSS) which was based on the presence or absence of symptoms, patient oxygen requirements, and whether or not the patient was admitted to the ICU/step down units. They again noted a positive trend between growth factor levels and increasing COVID severity. Because we were interested in the role of TGF-β1 in the pathogenesis of other diseases as well, we also compared TGF-β1 levels in patients with a prior history of disease including chronic lung disease, chronic kidney disease, chronic heart disease, pneumonia, high blood pressure, diabetes, previous strike, and abnormal chest x-ray upon ED admission. However, due to a limited sample size, a significant increase in TGF-β1 was only noted in the patients with a history of chronic kidney disease as compared to those without a history of chronic kidney disease. Others have reported that treatment of breast cancer cells with GLPG-0187 decreased TGF-β signaling. Thus, treatment with GLPG-0187 may especially benefit populations of patients with high levels of TGF-β1.

Because the inventors were interested in the concentrations of both active TGF-β1 and total TGF-β1, we next analyzed the patient plasma samples for active TGF-β1. They observed similar trends as described above and noted that active plasma TGF-β1 levels correlate with total TGF-β1 levels. A significant correlation was again noted between TGF-β1 plasma concentration and patient age. The inventors similarly noted higher levels of the growth factor in self-reported White and Hispanic or Latino populations, and notably lower levels in Black and Asian or Pacific Islander populations. When comparing active TGF-β1 levels between sexes, they again did not note a significant difference. Finally, a positive trend was noted between active TGF-β levels and increasing COVID severity, as determined by the CSS criteria.

In some embodiments, the subject has increased TGF-β1 levels. A subject having increased TGF-β1 levels has a higher level of TGF-β1 (active and/or total) than the normal TGF-β1 levels for the subject, which are known to those skilled in the art and can be determined by evaluating healthy control subjects. An increased TGF-β1 level can be about a 10%, 20%, 30%, 40%, 50%, 60%, 70%, 80%, 90%, 100%, 150%, or more than 200% increased level, compared with expected control levels.

The method includes administering to the subject a therapeutically effective amount of the integrin inhibitor. A number of integrin inhibitors are known to those skilled in the art. Examples of integrin inhibitors include vedolizumab, Integrin linked kinase ILK-IN-3, A-205804, A286982, RGD peptide, cilengitide, OSU-T315, Cilengitide trifluoroacetate, SB273005, cyclo(RGDyK), TR-14035, tirofiban, lifitegrast, ATN-161, tirofiban hydrochloride, pyrintegrin, leukadherin-1, and GLPG-0187. See Slack et al., Nat Rev Drug Discov., 21(1):60-78 (2022). Integrin inhibitors include selective integrin inhibitors and broad-spectrum integrin inhibitors.

In some embodiments, the integrin inhibitor is GLPG-0187. GLPG-0187 is a broad-spectrum integrin receptor antagonist that inhibits αᵥβ₁-integrin with an IC₅₀ of 1.3 nM, and is primarily known as an anticancer agent. See Horst et al., Neoplasia, 13(6):516-25 (2011). The structure of GLPG-0187 is shown below:

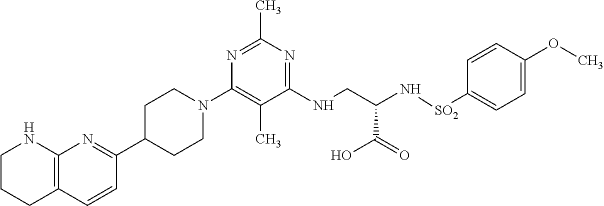 [SMILES: COc1ccc(S(=O)(=O)N[C@@H](CNc2nc(C)nc(N3CCC(c4ccc5c(n4)NCCC5)CC3)c2C)C(=O)O)cc1]

Treatment with GLPG-0187 inhibited pseudovirus infection in a dose-dependent manner in the D614G variant. In addition to D614G, several other SARS-CoV-2 pseudovirus variants were also tested including D614, N501Y, E484K, N501Y+E484K (N+E), N501Y+E484K+K417N (NEK), R685A. HSAE cells pre-treated with 1 μM GLPG-0187 for 3 hours followed by spin-infection with pseudovirus variants for 20 hours demonstrated inhibition of viral infection of each variant. To test inhibition of Beta and Delta variant pseudovirus infection, HSAE cells were pre-treated with 1 or 2 μM GLPG-0187 for 2 hours followed by spin-infection with pseudovirus. Pre-treatment with the integrin inhibitor resulted in the most significant decrease in pseudovirus infection by the Delta variant. The inventors conducted experiments with Omicron pseudovirus infection on HSAE cells with or without the integrin inhibitor GLPG-0187. The results suggest that Omicron pseudovirus was less capable of infecting the small airway epithelial cells than D614G or Delta variant pseudovirus. Nevertheless, the integrin inhibitor GLPG-0187 effectively blocked D614G, Delta and Omicron pseudovirus infection of HSAE cells.

In some embodiments, the method treats COVID-19 in a subject in need thereof, while in other embodiments the method prevents COVID-19 in a subject in need thereof.

In some embodiments, the method includes administering a mitogen-activated protein kinase (MEK) inhibitor to the subject. MEK inhibitors include broad-spectrum and selective MEK inhibitors. Examples of broad-spectrum MEK inhibitors include trametinib, PD0325901, selumetinib, U0126-EtOH, PD8059, and VS-6766, while examples of selective MEK inhibitors include cobimetinib and BIX 02189.

The inventors have previously demonstrated that MEKi compounds including VS-6766 reduce cellular expression of ACE2 and inhibit pseudovirus infection of multiple human cell types. Thus, they hypothesized that VS-6766 and GLPG-0187 could have an additive or synergistic inhibitory effect on pseudovirus infection of lung epithelial cells. To investigate this, they pre-treated HSAE cells with either 5 μM VS-6766 for 24 hours, 1 μM GLPG-0187 for 3 hours, or 5 μM VS-6766 for 24 hours followed by an additional 3 hours with GLPG-0187. After drug treatment, cells were spin-infected with the D614G pseudovirus for 20 hours. As expected, VS-6766 and GLPG-0187 single treatment inhibited pseudovirus infection when compared to the positive control. Combination treatment enhanced the inhibition of pseudovirus infection compared to single agent treatment with either VS-6766 or GLPG-0187.

In some embodiments, the MEK inhibitor is VS-6766. VS-6766, also known as R05126766 or Avutometinib, has the structure shown below:

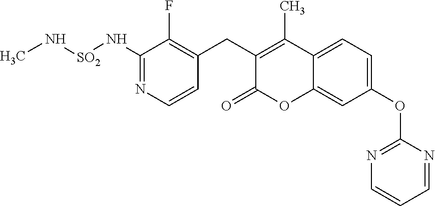 [SMILES: CNS(=O)(=O)Nc1nccc(Cc2c(C)c3ccc(Oc4ncccn4)cc3oc2=O)c1F]

wherein the integrin inhibitor is administered together with a pharmaceutically acceptable carrier.

As used herein, “a subject in need” refers to a subject who has, or has an increased risk, of developing COVID-19 infection, or an increased susceptibility to COVID-19 infection. For example, a subject may have increased risk for developing COVID-19 infection if they are known to have been exposed to COVID-19, or may have an increased susceptibility to COVID-19 infection if they are diabetic or have one or more known risk factors for COVID-19 infection, such as increased age, immunosuppression, male gender, or obesity. In some embodiments, the subject is a human subject.

### Formulations and Methods of Administration

In some embodiments, the integrin inhibitor (and in some embodiments, the MEK inhibitor) is administered together with a pharmaceutically acceptable carrier. The term “pharmaceutically acceptable”, when used in reference to a carrier, is meant that the carrier, diluent or excipient must be compatible with the other ingredients of the formulation and not deleterious to the recipient thereof.

When preparing the active compounds for oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitoneal administration, the active compounds may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

Formulations suitable for parenteral administration conveniently comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

### Example

Integrin/TGF-β1 inhibitor GLPG-0187 blocks SARS-CoV-2 Delta and Omicron pseudovirus infection of airway epithelial cells which could attenuate disease severity

The inventors have previously demonstrated the feasibility of a SARS-CoV-2 pseudovirus model system to evaluate the effects of drug treatment on viral infection. Zhou et al., Oncotarget, 11(46):4201-23 (2020). Here, they show that the pan-integrin inhibitor GLPG-0187 inhibits infection of multiple pseudovirus variants in HSAE cells, including the highly transmissible Delta variant which was the most prevalent strain as of August 2021 (Shiehzadegan et al., Clin Pract., 11(4):778-84 (2021)) and the Omicron variant which became the most prevalent in December 2021. This finding is clinically relevant as GLPG-0187 is in Phase I for treatment of solid tumors and has shown a favorable toxicity profile in patients. Cirkel et al., Invest New Drugs., 34(2):184-92 (2016). GLPG-0187 targets the integrins αvβ1, αvβ3, αvβ5, αvβ6, and α5β1, which in addition to allowing infection of the virus may play a potential role in SARS-CoV-2 pathogenesis by mediating activation of TGF-β, angiogenesis, lung injury, and inflammation. Carvacho et al., Clin Transl Immunology, 10(3):e1240 (2021).

The inventors previously demonstrated that treatment of HSAE cells with various MEK inhibitor (MEKi) compounds such as VS-6766 reduced cellular expression of ACE2 and inhibited pseudovirus infection. Zhou et al., ibid. Thus, we hypothesized that GLPG-0187 and VS-6766 may have an additive or synergistic inhibitory effect on pseudovirus infection. VS-6766 received FDA Breakthrough Therapy Designation in combination with defactinib for treatment of ovarian cancer in 2021, easing potential clinical translation.

The inventors previously reported that SARS-CoV-2 pathogenesis can lead to myriad changes in cytokine, chemokine, and growth factor profiles in patient plasma samples, and that these changes are associated with disease severity. Huntington et al., eLife, 10:e64958 (2021). They recognized in their previous study that COVID-19 disease severity was associated with macrophage activation syndrome. Integrins can activate TGF-β, a growth factor secreted as a latent complex, which plays a role in immune response, fibrosis, and viral replication. The TGF-β complex consists of three proteins including TGF-β, latency-associated protein (LAP), and an extracellular matrix-binding protein. LAP contains an RDG integrin-binding site which mediates activation of latent TGF-β via RGD-binding integrins. The chronic immune response observed with SARS-CoV-2 is believed to be mediated by TGF-β. Thus, it has been suggested that SARS-CoV-2 pathogenesis could be controlled via modulation of TGF-β. Chen, W. A., Int J Biol Sci., 16(11):1954-5 (2020).

The current study suggests that Omicron may less likely infect lower airway cells in the lung than other COVID-19 variants, and that integrin inhibitors have the potential to both prevent infection with SARS-CoV-2, including the Delta and Omicron variants, and to decrease TGF-β levels, resulting in a decrease in COVID-19 severity, hospitalization and death, especially in vulnerable and unvaccinated populations.

### Methods

### Cell Culture

HSAE cells (ATCC PCS-301-010) were cultured in Airway Epithelial Cell Basal Medium (ATCC PCS-300-030) supplemented with the Bronchial Epithelial Cell Growth Kit (ATCC PCS-300-040) at 37° C. in humidified atmosphere containing 5% CO₂.

### SARS-CoV-2 Pseudoviruses and Cell Entry Assays

The inventors developed a SARS-CoV-2 pseudovirus model system that uses pseudotyped SARS-CoV-2 viruses with a lentiviral core and a variety of SARS-CoV-2 spike protein variants on its envelope. To assess infectivity of normal human small airway epithelial (HSAE) cells, we used flow cytometry to quantify infected cells that express a fluorescence protein ZsGreen. A replication incompetent SARS-CoV-2 pseudovirus was generated using a lentiviral packaging system as previously described (Zhou et al., ibid). Briefly, 293FT cells (Invitrogen) at 75% confluency were co-transfected with the backbone vector pHAGE-fullEFla-Luciferase-IRES-ZsGreen, plasmids expressing lentiviral proteins Tat, Rev and Gag/Pol, and plasmids expressing D614 or D614G S protein (a gift from Dr. Hyeryun Choe, The Scripps Research Institute, Jupiter, FL), or S protein with N501Y, E484K, N501Y+E484K or N501Y+E484K+K417N mutations. An S protein expression plasmid construct containing all Beta variant (B.1.351) mutations and another S protein construct containing all Delta variant (B.1.617.2) mutations were gifts from Drs. Markus Hoffmann and Stefan Poehlmann (German Primate Center, Goettingen, Germany). An S protein expression plasmid construct containing all Omicron variant (B.1.1.529) mutations (Hoffman et al., Cell, 185(3):447-456.e11 (2022)) was custom-made by GenScript (Piscataway, NJ): pcDNA3.1(+)-SARS-CoV-2-Omicron-(6×His)-Spike (human codon). The variant S genes in the above constructs were sequenced to confirm all the corresponding mutations. A plasmid expressing VsVg protein instead of the S protein was used to generate a pantropic control lentivirus. Cell culture supernatants were collected, filtered, concentrated using ultra-centrifugation, aliquoted, and frozen at −80° C. Virus titer was determined using Lenti-X™ p24 Rapid Titration ELISA Kit (TaKaRa) and lentiviral particles were analyzed on an SDS-PAGE gel followed by Western blot to detect C-terminal FLAG-tagged S protein. HSAE cells were pre-treated with the integrin inhibitor GLPG-0187 (Galapagos NV, Mechelen, Belgium), MEK inhibitor VS-6766 (Verastem Oncology, Needham, MA), or both, for 2-27 hours. Following drug treatment, HSAE cells were spin-infected with SARS-CoV-2 pseudoviruses or a pantropic VsVg positive control lentivirus in a 12-well plate (931 g, 2 hours, 30° C. with 8 μg/ml polybrene). Analysis of ZsGreen+ cells was conducted by flow cytometry 20-24 hours after infection using a BD LSRII flow cytometer and FlowJo software.

### Human Plasma Samples

COVID-19 (+) human plasma samples were received from the Lifespan Brown COVID-19 Biobank at Rhode Island Hospital (Providence, RI, USA). All patient samples were deidentified but contained associated clinical information, as described. The IRB study protocol “Pilot Study Evaluating Cytokine Profiles in COVID-19 Patient Samples” did not meet the definition of human subjects research by either the Brown University or the Rhode Island Hospital IRBs.

### IRB/Oversight of Exemption for the Research

COVID-19 (+) and (−) human plasma samples were received from the Lifespan Brown COVID-19 Biobank from Brown University at Rhode Island Hospital (Providence, Rhode Island). All patient samples were deidentified but included the available clinical information as described. The IRB study protocol Pilot Study Evaluating Cytokine Profiles in COVID-19 Patient Samples did not meet the definition of human subjects research by either the Brown University or the Rhode Island Hospital IRBs. This is based on the fact that the project used deidentified specimens from a biobank with a determination that this project did not meet the definition of human subjects research based on specific criteria as described below. The original samples were collected at Rhode Island hospital by the Lifespan Brown COVID-19 Biobank through an IRB-approved protocol that involved informed consent that was used by the biobank. A human subjects determination form was completed for the Human Subjects Protection Program at Brown University.

### Cytokine Profiling

A Human Magnetic Luminex Performance Assay TGF-β1 Base Kit (Cat #LTGM100, R&D Systems, Inc., Minneapolis, MN) was run on a Luminex 200 Instrument (LX200-XPON-RUO, Luminex Corporation, Austin, TX) according to the manufacturer's instructions. Total TGF-β was quantified by activating patient samples with 1N HCl, neutralizing with 1.2N NaOH/0.5M HEPES, and then immediately assaying for TGF-β1. Active TGF-β was quantified without sample activation or neutralization prior to analysis.

### Statistical Analysis

A spearman's correlation was used to calculate statistical significance of scatter plots while the statistical significance between groups was determined using a One-way Anova followed by a post-hoc Tukey's multiple comparisons test. A two-tailed, unpaired student's t test was used to calculate statistical significance of pairs. The minimal level of significance was P<0.05. Following symbols * and ** represent, P<0.05 and P<0.01, respectively.

### Results

### Integrin Inhibition Decreases Infection of SARS-CoV-2 Pseudovirus Variants in Human Small Airway Epithelial Cells

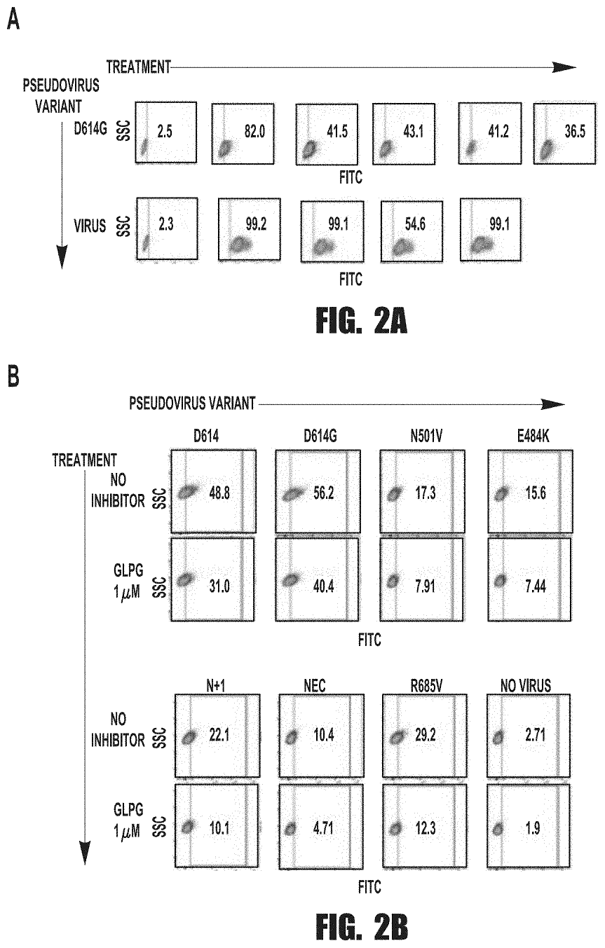

To test the inhibition of SARS-CoV-2 pseudovirus infection with the integrin inhibitor GLPG-0187, HSAE cells were pre-treated with 20 nM, 100 nM, 200 nM, or 1 μM GLPG-0187 for 2 hours followed by spin-infection with either a pseudovirus expressing the D614G spike protein variant or a VsVg positive control for 24 hours. Few ZsGreen+ cells were seen in the cells not treated with spin-infection, efficient viral infection was observed in cells treated with spin infection, and no effect of the inhibitor was observed on the VsVg positive control, as expected. Treatment with GLPG-0187 inhibited pseudovirus infection in a dose-dependent manner in the D614G variant (FIG. 2A). In addition to D614G, several other SARS-CoV-2 pseudovirus variants were also tested including D614, N501Y, E484K, N501Y+E484K (N+E), N501Y+E484K+K417N (NEK), R685A. HSAE cells pre-treated with 1 μM GLPG-0187 for 3 hours followed by spin-infection with pseudovirus variants for 20 hours demonstrated inhibition of viral infection of each variant (FIG. 2B). To test inhibition of Beta and Delta variant pseudovirus infection, HSAE cells were pre-treated with 1 or 2 μM GLPG-0187 for 2 hours followed by spin-infection with pseudovirus. Pre-treatment with the integrin inhibitor resulted in the most significant decrease in pseudovirus infection by the Delta variant (FIG. 2C). We conducted experiments with Omicron pseudovirus infection on HSAE cells with or without the integrin inhibitor GLPG-0187 (FIGS. 2D and E). The results suggest that Omicron pseudovirus was less capable of infecting the small airway epithelial cells than D614G or Delta variant pseudovirus, which is in agreement with a recent study by Meng et al. Meng et al., bioRxiv, 2021.12.17.473248 (2021). Nevertheless, the integrin inhibitor GLPG-0187 effectively blocked D614G, Delta and Omicron pseudovirus infection of HSAE cells.

### MEK Inhibitor Pre-Treatment Enhances Inhibition of Pseudovirus Infection by GLPG-0187 in Human Small Airway Epithelial Cells

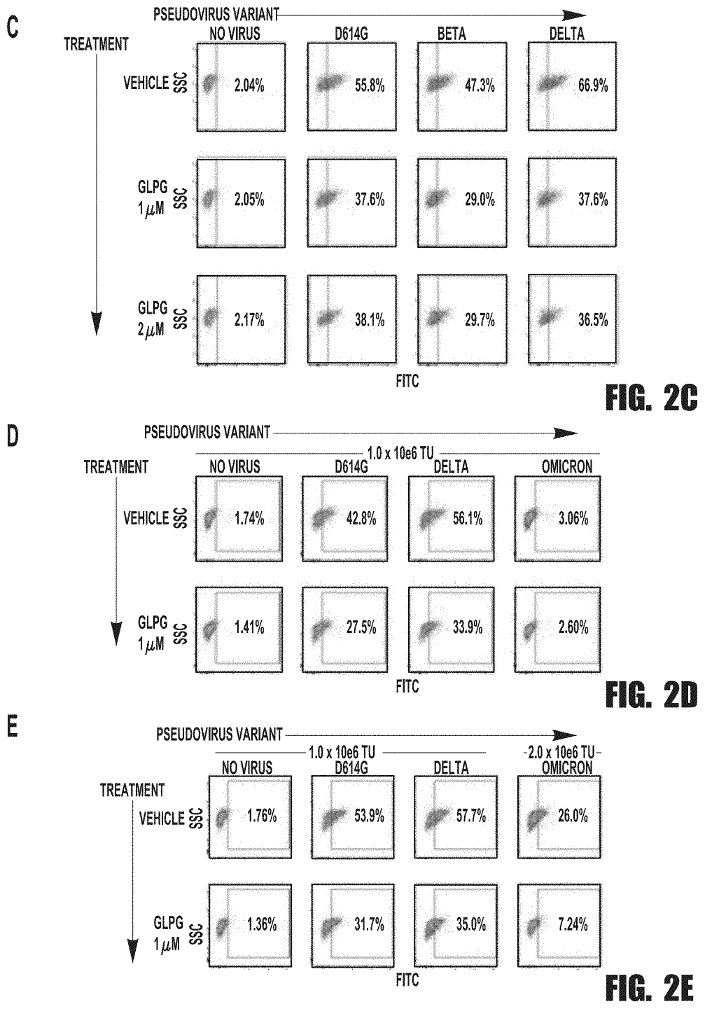

The inventors have previously demonstrated that MEKi compounds including VS-6766 reduce cellular expression of ACE2 and inhibit pseudovirus infection of multiple human cell types. Zhou et al., ibid. Thus, they hypothesized that VS-6766 and GLPG-0187 could have an additive or synergistic inhibitory effect on pseudovirus infection of lung epithelial cells. To investigate this, they pre-treated HSAE cells with either 5 μM VS-6766 for 24 hours, 1 μM GLPG-0187 for 3 hours, or 5 μM VS-6766 for 24 hours followed by an additional 3 hours with GLPG-0187. After drug treatment, cells were spin-infected with the D614G pseudovirus for 20 hours. As expected, VS-6766 and GLPG-0187 single treatment inhibited pseudovirus infection when compared to the positive control. Combination treatment enhanced the inhibition of pseudovirus infection compared to single agent treatment with either VS-6766 or GLPG-0187 (FIG. 3).

Plasma TGF-β1 Levels Correlate with Age, Race, and Number of Medications Administered Upon Presentation with COVID, but not with Sex

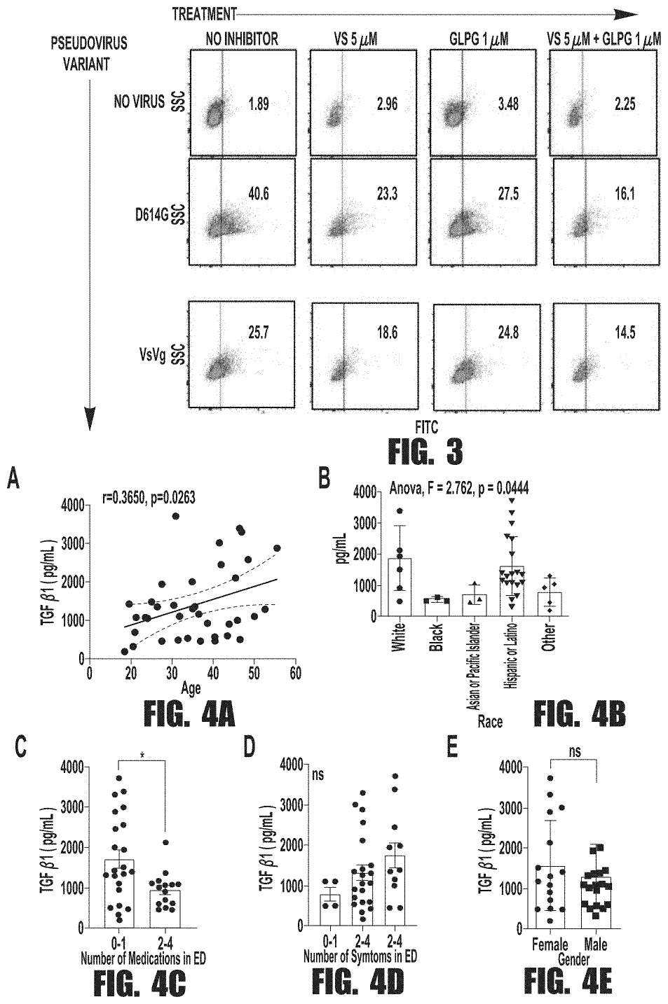

Because it has been previously shown that the chronic immune response observed with SARS-CoV-2 is mediated by TGF-β, the inventors sought to compare the levels of TGF-β1 in plasma samples from COVID (+) patients upon admission to the emergency department (ED). We chose to focus on TGF-β1, as opposed to TGF-02 and 3, since it had been previously shown that SARS-CoV-2 infection increased TGF-β1 expression in human epithelial cells and was a driver of lung fibrosis. Xu et al., Respiratory Research, 21(1):182 (2020). The levels of total TGF-β1 in COVID (+) plasma samples were analyzed and found a significant correlation between TGF-β1 concentration (pg/mL) and age (FIG. 4A). They also found significant variations in TGF-β1 concentrations depending on the patient's self-reported race or ethnicity, with notably higher levels of the growth factor in White and Hispanic or Latino populations, and notably lower levels in Black and Asian or Pacific Islander populations (FIG. 4B). The inventors next grouped patients by the number of medications they received upon disease presentation to the emergency department (FIG. 4C). Medications reported included ibuprofen, acetaminophen, bronchodilators (e.g., Albuterol), steroids (e.g., Prednisone), azithromycin, hydroxychloroquine, antibiotic, or other. We noticed statistically significant decreased plasma TGF-β1 concentrations in patients that received 2-4 medications in the Emergency Department (ED), as compared to patients who received 0-1. Next, they grouped patients by number of symptoms self-reported upon admission to the ED and noted a positive trend between TGF-β1 levels and the number of symptoms, although not significant (FIG. 4D). When comparing TGF-β1 levels between male and female, they did not note a significant difference (FIG. 4E).

The inventors also compared TGF-β1 levels in patients based on their COVID-19 Severity Score (CSS) (FIG. 4F) which was based on the presence or absence of symptoms, patient oxygen requirements, and whether or not the patient was admitted to the ICU/step down units (FIG. 4G). They again noted a positive trend between growth factor levels and increasing COVID severity. Because the inventors were interested in the role of TGF-β1 in the pathogenesis of other diseases as well, we also compared TGF-β1 levels in patients with a prior history of disease including chronic lung disease, chronic kidney disease, chronic heart disease, pneumonia, high blood pressure, diabetes, previous strike, and abnormal chest x-ray upon ED admission. However, due to a limited sample size, they only noted a significant increase in TGF-β1 in the patients with a history of chronic kidney disease as compared to those without a history of chronic kidney disease. Others have reported that treatment of breast cancer cells with GLPG-0187 decreased TGF-β signaling. Li et al., Breast Cancer Res., 17(1):28 (2015). Thus, treatment with GLPG-0187 may especially benefit populations of patients with high levels of TGF-β1.

Active Plasma TGF-β1 Levels Correlate with Total TGF-β1 Levels

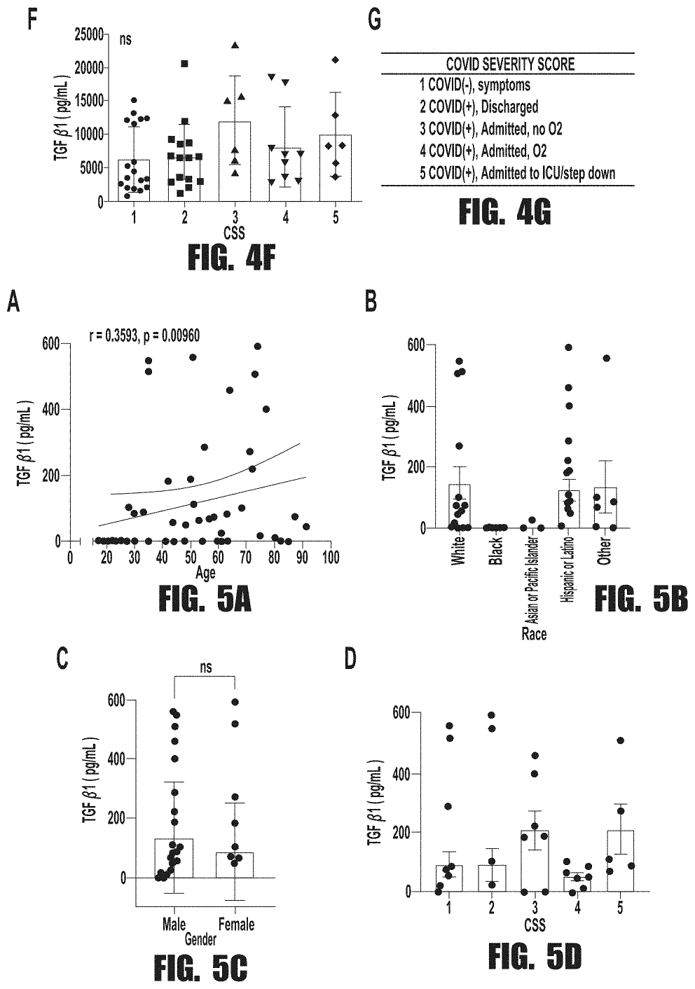

Because the inventors were interested in the concentrations of both active TGF-β1 and total TGF-β1, they next analyzed the patient plasma samples for active TGF-β1. Similar trends were observed as described above and it was noted that active plasma TGF-β1 levels correlate with total TGF-β1 levels (FIG. 5). The inventors again noted a significant correlation between TGF-β1 plasma concentration and patient age (FIG. 5A). They similarly noted higher levels of the growth factor in self-reported White and Hispanic or Latino populations, and notably lower levels in Black and Asian or Pacific Islander populations (FIG. 5B). When comparing active TGF-β1 levels between sexes, they again did not note a significant difference (FIG. 5C). Finally, the inventors again noted a positive trend between active TGF-β levels and increasing COVID severity, as determined by our CSS criteria (FIG. 5D).

### Discussion

SARS-CoV-2 remains a significant challenge in global health and new treatment options are needed, especially for vulnerable and unvaccinated populations. As of November 2021, the Delta variant accounted for more than 99% of COVID-19 cases and infection with this variant may result in an increased likelihood of hospitalization. Since then, the Omicron variant rapidly spread around the globe and became the dominant strain in many parts of the world. The current study suggests that integrin inhibition inhibits infection of multiple SARS-CoV-2 pseudovirus variants in HSAE cells, and that GLPG-0187 may be particularly effective in inhibiting infection by the Delta variant. As HSAE cells are thought to have very low expression of ACE2, alternative targets such as RGD-binding integrins may have particular value for treatment of COVID-19. Zhang et al., Am J Respir Crit Care Med., 202(2):219-29 (2020). The findings also suggest that combination treatment with a MEKi enhances this effect. In addition to inhibiting viral infection, it is possible that integrin inhibition could provide benefit to COVID-19 patients by reducing levels of active TGF-β, as integrins are a major regulator of TGF-β activation. Limited prior studies have reported that COVID-19 patients may have higher levels of TGF-β compared to healthy controls, which may mediate some of the complications in severe COVID-19 patients. Carvacho I, Piesche M., Clin Transl Immunology, 10(3):e1240 (2021).

Thus, benefit from GLPG-0187−/+ MEKi treatment in COVID-19 patients may be mediated through (1) inhibition of viral infection and (2) inhibition of TGF-β activation. Integrin inhibition may especially provide benefit to COVID-19 patient populations with particularly high levels of TGF-β such as elderly, White and Hispanic or Latino patients and patients who receive few medications in the ED, report a high number of symptoms, have a high CSS, and/or have a history of chronic kidney disease. Two limitations of this study include the small sample size of plasma samples from patients with COVID-19, as well as the lack of serial samples over time from the same patient.

Since its first identification in South Africa in November, 2021, the SARS-CoV-2 Omicron variant raised serious concerns of a significant reduction in efficacy of vaccines and monoclonal antibody treatments and an increased risk of reinfection due to numerous mutations in its spike protein, which is the antigenic target of infection- and vaccine-elicited antibodies against SARS-CoV-2. Currently, the Omicron variant is on track to outcompete the Delta variant as cases have soared to record highs in parts of Europe and now the U.S. according to the data released by Johns Hopkins University. A number of recent studies suggest that much of the Omicron variant's dominance comes down to its ability to evade the body's immune defenses. Hoffmann et al., Cell, 185(3):447-456.e11 (2022). However, earlier analyses of patients in South Africa suggest Omicron-infected individuals had a reduced risk of severe disease when compared to Delta-infected individuals. In the first findings on how the Omicron variant infects the respiratory tract, researchers from Hong Kong University reported that the virus multiplies 70 times faster in the bronchi than Delta and the original SARS-CoV-2 virus. In a potential clue regarding lower disease severity, they found that Omicron replication was less efficient in deeper lung tissue, more than 10 times lower than the original virus. In a recent study by Meng et al. (bioRxiv. 2021.12.17.473248 (2021)), the investigators found that despite three mutations predicted to favor spike S1/S2 cleavage, observed cleavage efficiency is substantially lower than for Delta, and Omicron pseudovirus entry into lower airway organoids and Calu-3 lung cells was thus impaired. In a recent study on mice and hamsters, Omicron produced less-damaging infections, often limited largely to the upper airway: the nose, throat and windpipe. The variant did much less harm to the lungs, where previous variants would often cause scarring and serious breathing difficulty. In our current study, we found that the Omicron pseudovirus was less capable of infecting the small airway epithelial cells than D614G or Delta variant pseudovirus, and that integrin inhibition effectively blocked D614G, Delta and Omicron pseudovirus infection of the small airway epithelial cells. Combined, these observations highlight that Omicron has gained immune evasion properties whilst compromising cell entry in lung cells, with possible implications for altered pathogenicity. In addition, targeting alternative viral infection routes such as integrin-mediated cell entry and dampening TGF-β1-mediated disease severity may have therapeutic implications, especially for vulnerable and unvaccinated populations.

It is possible that GLPG-0187 inhibits pseudovirus variant infection by an off-target effect on ACE2. It is also possible that (1) the virus may infect ACE2 negative cells by using RGD-binding integrins as an alternative receptor to ACE2 and/or (2) the RGD motif functions as a coreceptor that enhances viral infection via ACE2. The results demonstrate that GLPG-0187 inhibits pseudovirus entry, providing rationale for further investigation of integrin inhibitors as a potential therapy for COVID-19. Moreover, the inventors findings offer a combinatorial strategy combining an integrin inhibitor with a MEK inhibitor as a therapeutic strategy against COVID-19 including Delta and Omicron variants. These strategies could be further tested in clinical trials with particularly at risk patients with COVID-19 infection who are unvaccinated, immunosuppressed, or with risk factors such as comorbidities including cancer.

The complete disclosure of all patents, patent applications, and publications, and electronically available material cited herein are incorporated by reference. The foregoing detailed description and examples have been given for clarity of understanding only. No unnecessary limitations are to be understood there from. The invention is not limited to the exact details shown and described, for variations obvious to one skilled in the art will be included within the invention defined by the claims.

What is claimed is:

1. A method of treating or preventing COVID-19 in a subject in need thereof, comprising administering a therapeutically effective amount of an integrin inhibitor to the subject.

2. The method of claim 1, wherein the integrin inhibitor is GLPG-0187.

3. The method of claim 1, wherein the subject has increased TGF-β1 levels.

4. The method of claim 1, wherein the subject is a human subject.

5. The method of claim 1, wherein the COVID-19 is caused by Omicron or Delta SARS-CoV-2 infection.

6. The method of claim 1, wherein the method prevents COVID-19 in a subject in need thereof.

7. The method of claim 1, wherein the method treats COVID-19 in a subject in need thereof.

8. The method of claim 1, further comprising administering a mitogen-activated protein kinase (MEK) inhibitor to the subject.

9. The method of claim 7, wherein the MEK inhibitor is VS-6766.

10. the method of claim 1, wherein the integrin inhibitor is administered together with a pharmaceutically acceptable carrier.
